(19) United States
(12) Patent Application Publication
(10) Pub. No.: US 2019/0038154 A1
(43) Pub. Date: Feb. 7, 2019

(54) PULSE WAVE DETECTION DEVICE, BIOLOGICAL INFORMATION MEASURING DEVICE, WEARING AUXILIARY MEMBER OF PULSE WAVE DETECTION DEVICE

(71) Applicant: OMRON HEALTHCARE CO., LTD., Kyoto (JP)

(72) Inventors: Reiji FUJITA, Muko-shi (JP); Shinya TANAKA, Muko-shi (JP)

(21) Appl. No.: 16/158,462

(22) Filed: Oct. 12, 2018

Related U.S. Application Data
(63) Continuation of application No. PCT/JP2017/012942, filed on Mar. 29, 2017.

(30) Foreign Application Priority Data
Apr. 15, 2016 (JP) 2016-082370

(51) Int. Cl. A61B 5/021; A61B 5/00

(52) U.S. Cl. CPC A61B 5/02108; A61B 5/022; A61B 5/7235; A61B 5/681

(57) ABSTRACT

A pulse wave detector which is used while being attached to a wrist of a measurement subject includes: a body portion which includes a detecting section configured to detect a pulse wave from a radial artery of the measurement subject; a strip-like band which is configured to secure the body portion to the wrist; and an attachment auxiliary member which is configured to be attachable to and detachable from the band. In a secured state where the body portion is secured to the wrist with the band to which the attachment auxiliary member is attached, the attachment auxiliary member is contacted with a vicinity of an ulna or radius of the wrist.

### CROSS-REFERENCE TO RELATED APPLICATIONS

This is a Continuation of International Patent Application No. PCT/JP2017/012942 filed Mar. 29, 2017, which claims the benefit of Japanese Patent Application No. 2016-082370 filed Apr. 15, 2016. The disclosures of the prior applications are hereby incorporated by reference herein in their entireties.

### TECHNICAL FIELD

Aspects of the present invention relate to a pulse wave detector, a biometric information measurement device, and an attachment auxiliary member for a pulse wave detector.

### BACKGROUND ART

A biometric information measurement device is known that, in a state where a sensor is directly contacted with a living body portion through which an artery such as the radial artery in the wrist passes, can measure biometric information such as the heart rate, the pulse rate, or the blood pressure by using information detected by the sensor (for example, see Patent Literature 1 (JP-A-2008-168054) and Patent Literature 2 (JP-A-S51-041285)).

Patent Literature 1 discloses a biometric information measurement device which is attached to the wrist by using only a housing accommodating a sensor. In the housing of the biometric information measurement device, an opening for avoiding the ulna is disposed in a portion that is to be wound around the back side of the hand in a state where the device is attached to the wrist. This opening enables the attachment state of the device to the wrist, to be stably maintained.

Patent Literature 2 discloses a biometric information measurement device in which both end portions of a band portion that is to be wound around the wrist are divided into three parts, and one and other ends of the divided band parts are enabled to be respectively secured.

In the biometric information measurement device of Patent Literature 1, it is necessary to contrive the material and shape of the housing, and therefore, the production cost of the device is increased. A configuration where the housing is secured to the wrist by using a band is effective for suppressing the production cost. In the wrist, however, there is a portion where the ulna protrudes. When the band is to be fastened, therefore, the band hits the ulna, and hence there arises a possibility that the feeling and easiness of attachment of the device may be impaired, or after attachment, displacement of the position of the device may occur.

When a highly stretchable material is used in the band, the feeling and easiness of attachment of the device can be improved. However, in the case of a band having a high stretchability, after the device is secured to the wrist, there arises a possibility that displacement of the position of the sensor portion may be caused by stretching or contraction of the band portion, and therefore, it is difficult to accurately measure biometric information.

In the biometric information measurement device of Patent Literature 2, sensors are secured to the divided parts of the band, respectively, thereby enabling the pressing positions of the sensors to be adjustable. However, the sensors are disposed on the band itself, and therefore, there is a high possibility that the positions of the sensors may be displaced by a motion of the hand. As a result, the device cannot accurately measure biometric information.

The above-described problems are not limited in a biometric information measurement device which detects a pressure pulse wave by using a pressure sensor or the like, and similarly occur also in a biometric information measurement device in which, for example, a volume pulse wave is detected by using a photoelectric sensor.

### SUMMARY

Embodiments of the present invention address the above disadvantages and other disadvantages not described above. However, the present invention is not required to overcome the disadvantages described above, and thus, an exemplary embodiment of the present invention may not overcome any of the problems described above.

A pulse wave detector according to an embodiment of the present invention is used while being attached to a wrist of a measurement subject and includes: a body portion which includes a detecting section configured to detect a pulse wave from a radial artery of the measurement subject; a strip-like band which is configured to secure the body portion to the wrist; and an attachment auxiliary member which is configured to be attachable to and detachable from the band, wherein in a secured state where the body portion is secured to the wrist with the band to which the attachment auxiliary member is attached, the attachment auxiliary member is contacted with a vicinity of an ulna or radius of the wrist.

A biometric information measurement device according to an embodiment of the present invention includes: the above pulse wave detector; and a biometric information calculating section which is configured to calculate biometric information based on the pulse wave detected by the pulse wave detector.

An attachment auxiliary member according to an embodiment of the present invention is for a pulse wave detector including a body portion which includes a detecting section configured to detect a pulse wave from a radial artery of a wrist of a measurement subject; and a strip-like band which is configured to secure the body portion to the wrist, wherein the attachment auxiliary member is configured to be attachable to and detachable from the band, and wherein in a secured state where the body portion is secured to the wrist with the band to which the attachment auxiliary member is attached, the attachment auxiliary member is contacted with a vicinity of an ulna or radius of the wrist.

### BRIEF DESCRIPTION OF DRAWINGS

The above and other aspects of the present invention will become more apparent and more readily appreciated from the following description of embodiments of the present invention taken in conjunction with the attached drawings.

FIG. 1 is a perspective view schematically showing the external configuration of a biometric information measurement device 100 of an embodiment of the present invention.

FIG. 2 is a side view of the biometric information measurement device 100 shown in FIG. 1, as seen from the side of the elbow of the left hand of a measurement subject (the central side of the measurement subject).

FIG. 3 is a plan view schematically showing a band 40 to which an attachment auxiliary member 30 is attached.

FIGS. 4A and 4B are perspective views schematically showing the external configuration of the attachment auxiliary member 30.

FIGS. 5A and 5B are side views of the attachment auxiliary member 30.

FIGS. 6A and 6B are perspective views schematically showing the external configuration of an attachment auxiliary member 50 which is a modification of the attachment auxiliary member 30.

FIGS. 7A and 7B are perspective views schematically showing the external configuration of an attachment auxiliary member 30A which is a modification of the attachment auxiliary member 30 of the biometric information measurement device 100 shown in FIG. 1.

FIG. 8 is a view showing a modification of the attachment auxiliary member 30 of the biometric information measurement device 100 shown in FIG. 1.

FIG. 9 is a view showing a modification of an attachment position of the attachment auxiliary member 30 of the biometric information measurement device 100 shown in FIG. 1.

### DESCRIPTION OF EMBODIMENTS

Hereinafter, embodiments of the present invention will be described with reference to the drawings.

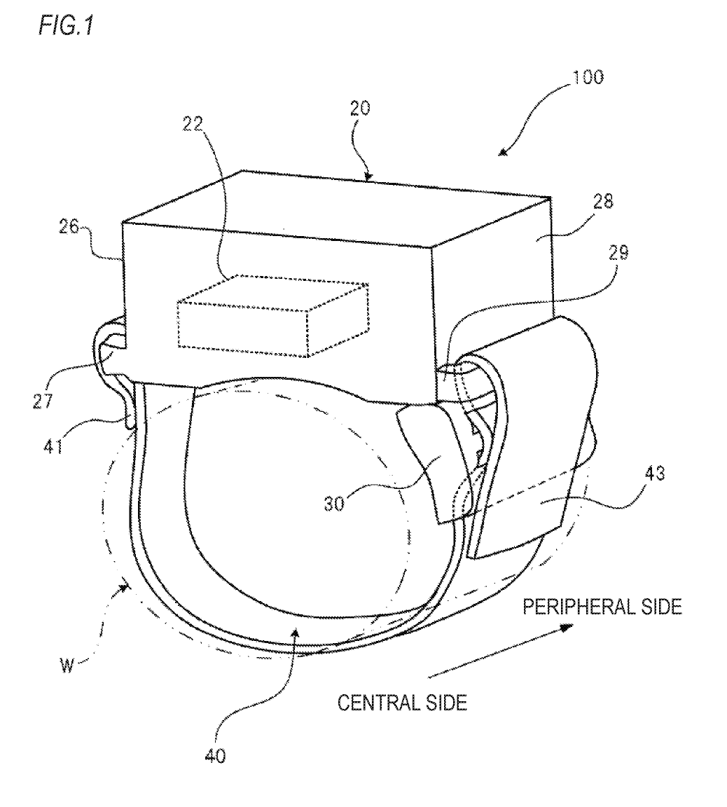

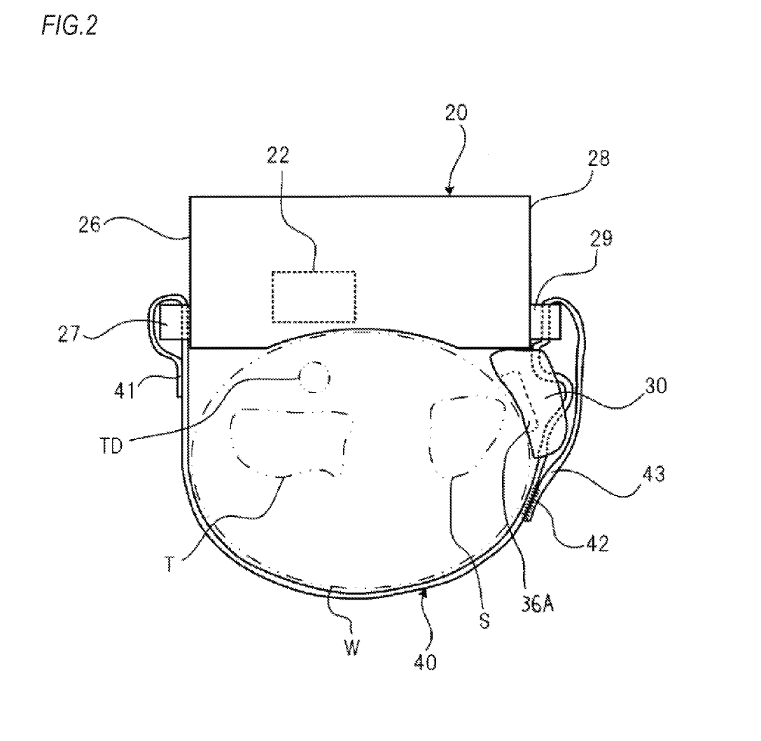

FIG. 1 is a perspective view schematically showing the external configuration of a biometric information measurement device 100 of an embodiment of the present invention. As shown in FIG. 1, the biometric information measurement device 100 is used while being attached to the wrist W of the left hand of a measurement subject. FIG. 2 is a side view of the biometric information measurement device 100 shown in FIG. 1, as seen from the side of the elbow of the left hand of the measurement subject (the central side of the measurement subject). Although the example in which the biometric information measurement device 100 is attached to the wrist of the left hand will be described, the biometric information measurement device 100 can be attached also to the wrist of the right hand.

The biometric information measurement device 100 has: a housing 20 which is formed of a metal or a resin; a strip-like band 40 which is a member for securing the housing 20 to the wrist W; and an attachment auxiliary member 30 for assisting the attachment of the housing 20 to the wrist W.

The housing 20 includes a pulse wave detecting section 22 which can detect a pulse wave (a pressure pulse wave or a volume pulse wave) from the radial artery TD that extends along the radius T in the wrist W of the measurement subject. The housing 20 constitutes the body portion of the biometric information measurement device 100. The body portion of the biometric information measurement device 100 may be configured only by the housing 20, or have a configuration where the housing 20 and a sub-housing incorporating batteries or the like are coupled to each other (a configuration where the portion is formed by a plurality of housings).

The pulse wave detecting section 22 includes at least a sensor which can output a pulse wave as a signal. For example, the pulse wave detecting section 22 has a pressure sensor, and a pressing mechanism which presses it against the skin, and detects a pressure pulse wave by using the pressure sensor. Alternatively, the pulse wave detecting section 22 has a photoelectric sensor, and detects a volume pulse wave from a signal detected by the photoelectric sensor.

In the example of FIG. 1, the housing 20 is a member having an approximately box-like shape, and includes the pulse wave detecting section 22, and a biometric information calculating section (not shown) which calculates biometric information such as the heart rate, the pulse rate, or the blood pressure value based on the pulse wave detected by the pulse wave detecting section 22.

The biometric information calculating section may be disposed in an apparatus other than the biometric information measurement device 100. Namely, the housing 20 (the body portion) of the biometric information measurement device 100 may need to have at least the pulse wave detecting section 22. In this case, the biometric information measurement device 100 functions as the pulse wave detector.

The surface of the housing 20 which, in the case where the biometric information measurement device 100 is attached to the wrist W, is opposed to the wrist W constitutes a detection surface. The detection surface may have an approximately flat shape, or a part or the whole of the surface may be curved so as to extend along the outer shape of the wrist W. The pulse wave detecting section 22 is disposed in a position which is opposed to the radial artery TD in a state where the detection surface is opposed to the wrist W.

A first engaging portion 29 is disposed on a first end portion 28 (in the example of FIGS. 1 and 2, an end surface of the housing 20 on the side of the ulna S) which is one of both end portions of the housing 20 on the side of the ulna S in the circumferential direction of the wrist W.

A second engaging portion 27 is disposed on a second end portion 26 (in the example of FIGS. 1 and 2, an end surface of the housing 20 on the side of the radius T) which is one of the end portions of the housing 20 on the side of the radius T in the circumferential direction of the wrist W.

The first engaging portion 29 and the second engaging portion 27 are used for causing the band 40 to engage with the housing 20. Each of the first engaging portion 29 and the second engaging portion 27 has a shape that has a hole portion into which the band 40 can be inserted.

The band 40 is wound around the wrist W in a state where a basal end portion 41 is engaged with the second engaging portion 27 of the housing 20 (routed so as to form a state where the wrist W is sandwiched between the band 40 and the housing 20), thereby securing the housing 20 to the wrist W while maintaining the state where the pulse wave detecting section 22 is opposed to the radial artery TD.

For example, the band 40 is a member which is lower in rigidity than the housing 20. As the material of the band 40, for example, cloth, leather, rubber, thin resin, or the like may be used.

The basal end portion 41 which constitutes one end of the band 40 in the longitudinal direction is engaged in a state where the tip end portion is folded back in the second engaging portion 27 which is disposed on the second end portion 26.

A tip end portion 43 which constitutes the other end of the band 40 in the longitudinal direction is engaged in a state where the tip end portion is folded back in the first engaging portion 29 which is disposed on the first end portion 28.

Specifically, the tip end portion 43 of the band 40 is inserted into the hole portion of the first engaging portion 29, in a direction from the side of the detection surface. The tip end portion 43 which has passed through the hole portion is folded back in an approximately U-like shape in a direction to be separated from the housing 20. A hook and loop fastener 42 is formed on the surface of the non-folded back part of the tip end portion 43 and directed in the direction opposite to the housing 20, and the surface of the folded back part of the tip end portion 43 and directed to the housing 20. Because of the hook and loop fastener 42, the folded back part of the tip end portion 43 is detachably engaged with the band 40.

The hook and loop fastener 42 is an engagement member for causing the tip end portion 43 to detachably engage with the band 40. A configuration may be employed where the tip end portion 43 is detachably engaged with the band 40 by using a concave portion and a convex portion which can be fitted thereinto.

The attachment auxiliary member 30 is a member which is configured so as to be attachable to and detachable from the band 40. Specifically, the attachment auxiliary member 30 has a through hole through which the band 40 can be passed. While the band 40 is passed through the through hole, the attachment auxiliary member 30 is attached to the band 40. Preferably, the attachment auxiliary member 30 is configured by a material which allows the band 40 to be easily passed therethrough (for example, wood, metal, or resin).

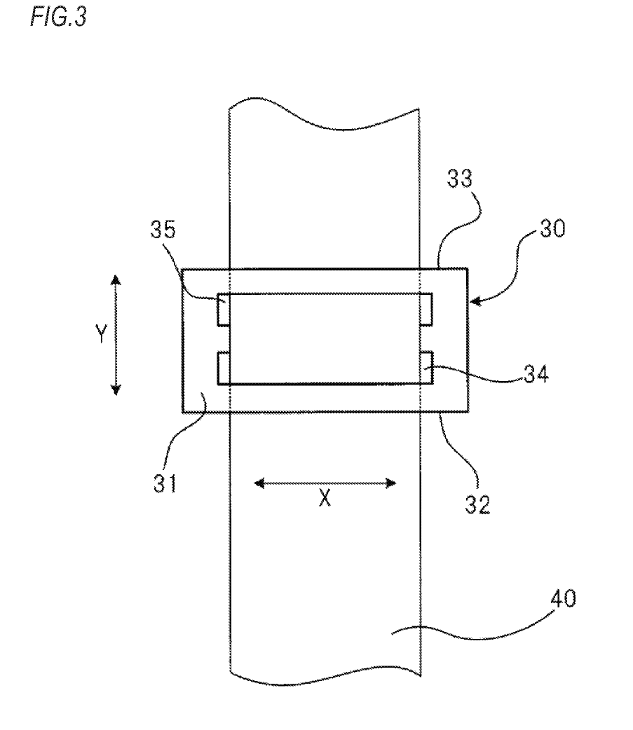

The configuration of the attachment auxiliary member 30 will be described in detail later with reference to FIGS. 3 to 5.

FIG. 3 is a plan view schematically showing the band 40 to which the attachment auxiliary member 30 is attached. The direction Y shown in FIG. 3 indicates the longitudinal direction of the band 40. The direction X shown in FIG. 3 indicates the short-side direction perpendicular to the longitudinal direction of the band 40.

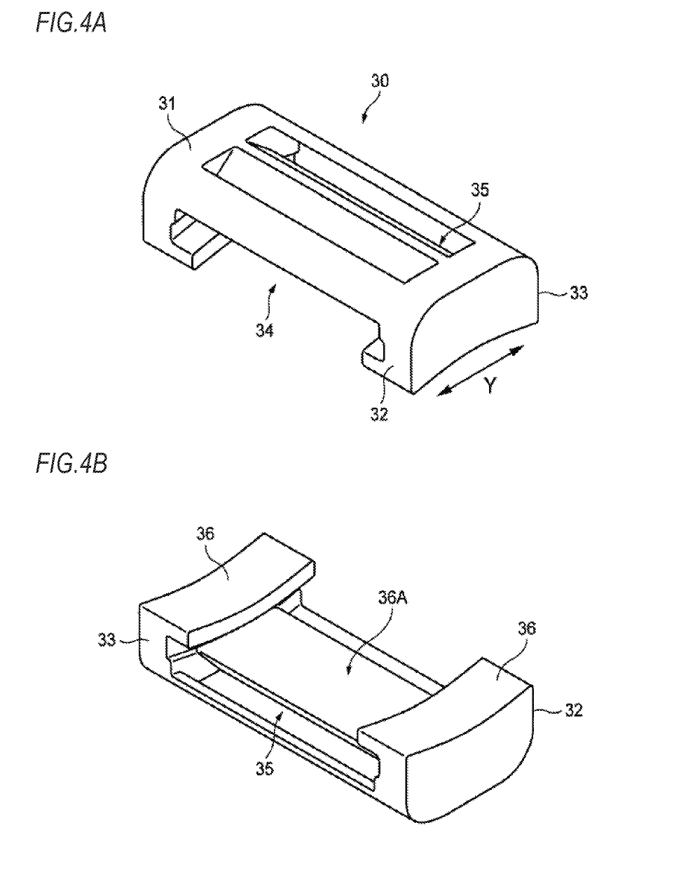

FIGS. 4A and 4B are perspective views schematically showing the external configuration of the attachment auxiliary member 30. FIG. 4A is a perspective view of the attachment auxiliary member 30, as seen from the side opposite to that opposed to the wrist W in a state where the housing 20 is secured to the wrist W by the band 40 to which the attachment auxiliary member 30 is attached (hereinafter, referred to as the secured state). FIG. 4B is a perspective view of the attachment auxiliary member 30, as seen from the side opposed to the wrist W in the secured state.

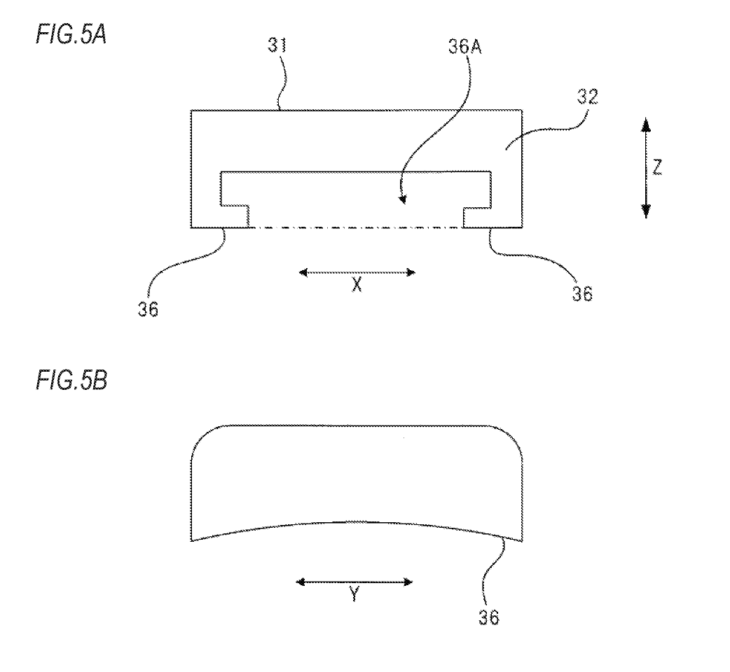

FIGS. 5A and 5B are side views of the attachment auxiliary member 30. FIG. 5A is a view of the attachment auxiliary member 30, as seen in the direction Y in FIG. 3. FIG. 5B is a view of the attachment auxiliary member 30, as seen in the direction X in FIG. 3. In FIG. 5, the direction Z (the direction in which the member is pressed against the skin in the secured state) which is perpendicular to both the directions X and Y shown in FIG. 3.

As shown in FIG. 4, the general shape of the attachment auxiliary member 30 is similar to a shape which is obtained by dividing a column into halves. In the front surface 31 of the attachment auxiliary member 30, a through hole 34 which reaches one side surface 32 of the attachment auxiliary member 30 in the direction Y, and a through hole 35 which reaches the other side surface 33 of the attachment auxiliary member 30 in the direction Y are formed. The band 40 which is inserted into the through hole 34 is passed through the through hole 35, and then pulled out from the side surface 33. Accordingly, the attachment auxiliary member 30 is attached to the band 40 in a state where the member is movable in the direction Y.

The rear surface 36 which is opposite to the front surface 31 of the attachment auxiliary member 30 is a portion which is opposed to the wrist W of the measurement subject in the secured state. A concave portion 36A is formed in the rear surface 36. The concave portion 36A has a size which allows the protrusion of the ulna S in the wrist W of the measurement subject to be received.

In the secured state, the attachment auxiliary member 30 is used while being placed in a position which is contacted with the vicinity of the ulna S in the wrist W. The vicinity of the ulna S in the wrist W means a range where the ulna S can be touched with the finger.

The existence of the concave portion 36A enables the protrusion of the ulna S to be received in the concave portion 36A even in a state where the attachment auxiliary member 30 is contacted with the skin of the vicinity of the ulna S. Therefore, interference between the protrusion of the ulna S and the attachment auxiliary member 30 can be prevented from occurring, and the attachability of the biometric information measurement device 100 can be improved.

As shown in FIG. 5A, the rear surfaces 36 of both end portions of the attachment auxiliary member 30 in the direction X are identical with each other in position in the the direction Z.

As shown in FIG. 5B, the rear surfaces 36 which are in contact with the wrist W in the secured state are curved surfaces that are curved along the circumferential direction of the wrist W. According to the configuration, the contactability between the rear surfaces 36 and the wrist W can be made satisfactory, and the attachability of the biometric information measurement device 100 can be improved.

An example of a method of attaching the thus configured biometric information measurement device 100 to the wrist W will be described.

As shown in FIG. 3, firstly, the measurement subject attaches the attachment auxiliary member 30 to the band 40 such that the rear surfaces 36 of the attachment auxiliary member 30 are directed to the skin. Then, the measurement subject inserts the tip end portion 43 of the band 40 into the hole portion of the first engaging portion 29 of the housing 20 from the side of the detection surface. In this state, the measurement subject finely adjusts the position of the attachment auxiliary member 30 by moving the attachment auxiliary member 30 in the longitudinal direction of the band 40, so that the protrusion of the ulna S enters the concave portion 36A of the attachment auxiliary member 30. After the adjustment, the measurement subject folds back and pulls the tip end portion 43 which has passed through the hole portion of the first engaging portion 29, thereby adjusting the degree of fastening of the band 40. Finally, the measurement subject causes the tip end portion 43 to engage with the band 40 through the hook and loop fastener 42, thereby completing the attachment of the biometric information measurement device 100.

According to the biometric information measurement device 100, as described above, a pressure which is applied from the protrusion of the ulna S to the band 40 can be absorbed by the concave portion 36A of the attachment auxiliary member 30. Therefore, a situation where the ulna S interferes the fastening of the band 40 to worsen the feeling and easiness of attachment of the biometric information measurement device 100 does not occur, and the feeling and easiness of attachment can be improved. Moreover, a further situation where, after attachment of the biometric information measurement device 100, the position of the device is displaced can be prevented from occurring. According to the biometric information measurement device 100, the material of the band 40 is not limited. Therefore, a material having an ideal stretchability for securing of the housing 20 can be employed, displacement of the position of the device can be prevented from occurring, and the accuracy of detection of a pulse wave can be improved.

According to the biometric information measurement device 100, the attachment auxiliary member 30 is placed between the wrist W and the band 40. Therefore, even in the case where a gap is formed between the wrist W and the band 40, the gap is filled by the attachment auxiliary member 30. Accordingly, the biometric information measurement device 100 can be closely contacted with the wrist W, and it is possible to prevent displacement of the position of the device from occurring after attachment of the biometric information measurement device 100. As a result, the accuracy of detection of a pulse wave can be improved.

According to the biometric information measurement device 100, the rear surfaces 36 of the attachment auxiliary member 30 which are in contact with the wrist W in the secured state are curved along the circumferential direction of the wrist W. Therefore, the close contactness between the attachment auxiliary member 30 and the wrist W can be improved, and the feeling of attachment of the biometric information measurement device 100 can be improved.

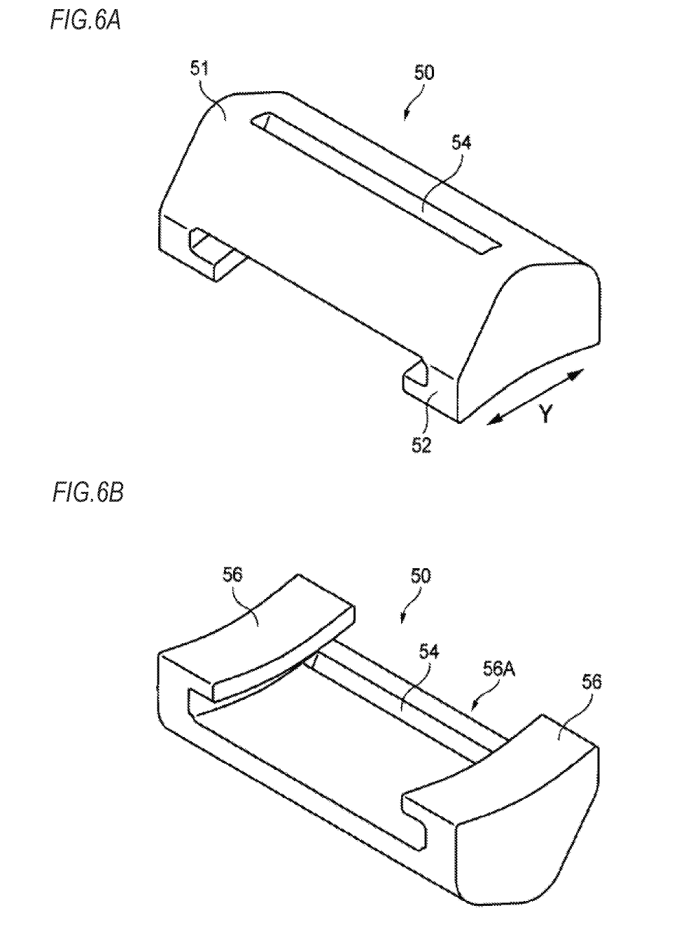

The configuration of the attachment auxiliary member 30 shown in FIG. 3 is an example, and not limited thereto. For example, the attachment auxiliary member 30 may have the configuration such as shown in FIGS. 6A and 6B.

FIGS. 6A and 6B are perspective views schematically showing the external configuration of an attachment auxiliary member 50 which is a modification of the attachment auxiliary member 30.

The general shape of the attachment auxiliary member 50 has a shape similar to that which is obtained by dividing a column into halves. In the front surface 51 of the attachment auxiliary member 50, a through hole 54 which reaches a side surface 52 of the attachment auxiliary member 50 in the direction Y is formed. The band 40 which is inserted into the through hole 54 from the side of the side surface 52 is pulled out from the side of the front surface 51. Therefore, the attachment auxiliary member 50 is attached to the band 40 in a state where the member is movable in the direction Y.

The rear surface 56 which is opposite to the front surface 51 of the attachment auxiliary member 50 is a portion which is opposed to the wrist W of the subject in the secured state. A concave portion 56A is formed in the rear surface 56. The concave portion 56A has a size which allows the protrusion of the ulna S in the wrist W of the measurement subject to be inserted. The concave portion 56A functions also as a part of the through hole 54.

The above-described effects can be attained also in the case where the attachment auxiliary member 50 shown in FIGS. 6A and 6B is used in place of the attachment auxiliary member 30.

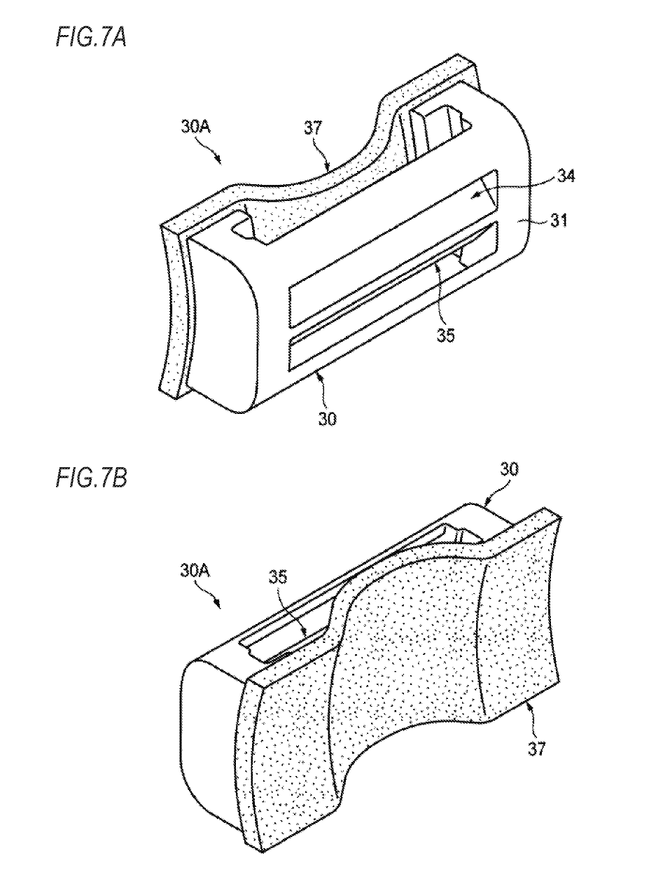

FIGS. 7A and 7B are perspective views schematically showing the external configuration of an attachment auxiliary member 30A which is a modification of the attachment auxiliary member 30 of the biometric information measurement device 100 shown in FIG. 1.

The attachment auxiliary member 30A shown in FIG. 7 has a configuration where a planar elastic member 37 is secured to the rear surface 36 of the attachment auxiliary member 30 by an adhesive material or the like.

The elastic member 37 is secured to the rear surface 36 while covering the concave portion 36A of the attachment auxiliary member 30, and in a portion overlapping with the concave portion 36A, has a shape which is recessed toward the concave portion 36A. Because of the recessed shape, a configuration is formed where the insertion of the protrusion of the ulna S into the concave portion 36A is not impeded. In the case where a material having a high stretchability is used as the elastic member 37, the recess is not essential.

The front surface of the elastic member 37 which is on the rear surfaces 36 of the attachment auxiliary member 30 follows the curved shape of the rear surfaces 36 to be formed as a curved surface.

Since the elastic member 37 is a portion which is to be contacted with the skin, the feeling of attachment of the biometric information measurement device 100 is considered, and the raw material which is lower in rigidity than the attachment auxiliary member 30 is selected as the elastic member 37. For example, rubber, sponge, or the like can be used as the elastic member 37.

According to the attachment auxiliary member 30A shown in FIG. 7, the portion which is to be contacted with the skin is the elastic member 37. Therefore, the contact between the attachment auxiliary member 30A and the skin can be satisfactorily performed, and the feeling of attachment of the biometric information measurement device 100 can be improved.

When a material which exerts a large frictional force with respect to the skin is used as the elastic member 37, an anti-slip function can be added to the attachment auxiliary member 30A, and it is possible to prevent displacement of the position of the biometric information measurement device 100 from occurring after attachment of the device.

The attachment auxiliary member 30 which has been described with reference to FIGS. 3 to 5 has the configuration where, as shown in FIG. 5A, the rear surfaces 36 of the both end portions in the direction X have the same position in the direction Z. Preferably, the member is configured so that the Z-direction positions of the rear surfaces 36 of the both end portions are different from each other.

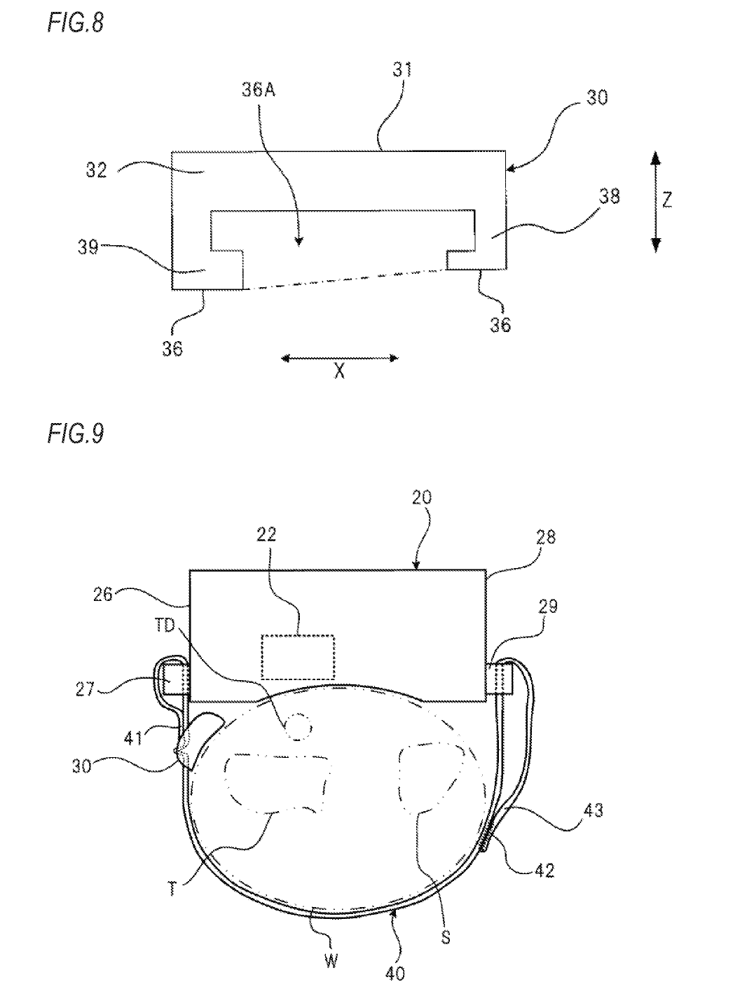

FIG. 8 is a view showing a modification of the attachment auxiliary member 30 of the biometric information measurement device 100 shown in FIG. 1. FIG. 8 is a view showing the attachment auxiliary member 30, as seen in the direction Y shown in FIG. 3.

In the attachment auxiliary member 30 shown in FIG. 8, the rear surface 36 of the end portion (peripheral side end portion) 39 which is placed on the peripheral side in the direction X in the secured state, and the rear surface 36 of the end portion (central side end portion) 38 which is placed on the central side in the direction X in the secured state are different in a position in the direction Z.

When an arbitrary position which is on the side of the front surface 31 with respect to the rear surface 36 of the central side end portion 38 in the direction Z is set as a reference position, specifically, the Z-direction position of the rear surface 36 of the peripheral side end portion 39 is remoter from the reference position than the Z-direction position of the rear surface 36 of the central side end portion 38.

The wrist W gradually becomes thinner as approaching from the central side toward the periphery. Therefore, when the configuration shown in FIG. 8 is employed, it is possible to prevent the attachment auxiliary member 30 from being inclined in the direction Z because of the difference in diameter of the wrist. Consequently, the band 40 can be prevented from being twisted, and the attachability of the biometric information measurement device 100 can be improved.

The attachment auxiliary member 30 of the biometric information measurement device 100 may be enabled to be attached to and detached from the band 40, by using an engagement member such as a hook and loop fastener. In this case, a hook and loop fastener disposed on a surface of the band 40 which is opposed to the wrist, and that disposed on the front surface 31 of the attachment auxiliary member 30 are coupled to each other, whereby the attachment auxiliary member 30 is attached to the band 40.

The attachment auxiliary member 30 may be used while being attached to a position which is contacted with the vicinity of the radius T in the wrist W in the secured state. As shown in FIG. 9, for example, the attachment auxiliary member 30 may be attached to the vicinity of the second engaging portion 27. In the case of the attachment state shown in FIG. 9, the rear surface of the attachment auxiliary member 30 is contacted with the vicinity of the radius T in the wrist W. The vicinity of the radius T in the wrist W means a range of the wrist W where the radius T can be touched with the finger.

In this case, the concave portion 36A has a size which allows the protrusion of the radius T to be received, and therefore, even in the state where the attachment auxiliary member 30 is in contact with the skin in the vicinity of the radius T, the protrusion of the radius T can be received in the concave portion 36A. Consequently, interference between the protrusion of the radius T and the attachment auxiliary member 30 can be prevented from occurring, and the attachability of the biometric information measurement device 100 can be improved.

In the above description, although the attachment auxiliary member 30 has the concave portion 36A in the rear surface, the concave portion 36A is not essential. In the case where the material constituting the rear surface of the attachment auxiliary member 30 is stretchable and has a certain thickness, or in the case where, as shown in FIGS. 7A and 7B, an elastic member which is stretchable, and which has a certain thickness is fixed to the rear surface of the attachment auxiliary member 30, for example, the material or the elastic member can absorb a pressure due to the protrusion of the ulna or the radius, even when the concave portion 36A is not disposed. Therefore, interference between a protrusion of a bone and the attachment auxiliary member 30 can be prevented from occurring.

The above-disclosed embodiment should be considered in all respects to be illustrative and not restrictive. The scope of the present invention is represented by the appended claims rather than the foregoing description, and all changes within the meaning and range of equivalents thereof are intended to be covered therein.

As described above, the following matters are disclosed in the specification.

The disclosed pulse wave detector is used while being attached to a wrist of a measurement subject and includes: a body portion which includes a detecting section configured to detect a pulse wave from a radial artery of the measurement subject; a strip-like band which is configured to secure the body portion to the wrist; and an attachment auxiliary member which is configured to be attachable to and detachable from the band, wherein in a secured state where the body portion is secured to the wrist with the band to which the attachment auxiliary member is attached, the attachment auxiliary member is contacted with a vicinity of an ulna or radius of the wrist.

In the disclosed pulse wave detector, the attachment auxiliary member is formed with a concave portion configured to receive a protrusion of the ulna or radius of the wrist in a portion which is opposed to the wrist in the secured state.

In the disclosed pulse wave detector, a surface of the attachment auxiliary member to be contacted with the wrist is curved along a circumferential direction of the wrist.

In the disclosed pulse wave detector, contact surfaces with the wrist of both end portions of the attachment auxiliary member in a short-side direction of the band in a state of being attached to the band are different in position in a direction perpendicular to both the short-side and longitudinal directions of the band.

In the disclosed pulse wave detector, a surface of the attachment auxiliary member to be contacted with the wrist is covered by an elastic member.

The disclosed biometric information measurement device includes: the above pulse wave detector; and a biometric information calculating section which is configured to calculate biometric information based on the pulse wave detected by the pulse wave detector.

The disclosed attachment auxiliary member for a pulse wave detector including a body portion which includes a detecting section configured to detect a pulse wave from a radial artery of a wrist of a measurement subject; and a strip-like band which is configured to secure the body portion to the wrist, is configured to be attachable to and detachable from the band, and in a secured state where the body portion is secured to the wrist with the band to which the attachment auxiliary member is attached, the attachment auxiliary member is contacted with a vicinity of an ulna or radius of the wrist.

In the disclosed attachment auxiliary member, a concave portion which is configured to receive a protrusion of the ulna or radius of the wrist is formed in a portion which is opposed to the wrist in the secured state.

In the disclosed attachment auxiliary member, a surface of the attachment auxiliary member to be contacted with the wrist is curved along a circumferential direction of the wrist.

In the disclosed attachment auxiliary member, contact surfaces with the wrist of both end portions of the attachment auxiliary member in a short-side direction of the band in a state of being attached to the band are different in position in a direction perpendicular to both the short-side and longitudinal directions of the band.

In the disclosed attachment auxiliary member, a surface which is to be contacted with the wrist is covered by an elastic member.

Accordingly, it is possible to provide a pulse wave detector in which the feeling and easiness of attachment to the wrist can be improved, and which can accurately detect a pulse wave, a biological information measurement device including the detector, and an attachment auxiliary member which is to be used in a pulse wave detector.

The present invention is convenient and effective particularly in application to a blood pressure monitor or the like.

Although the present invention has been described with reference to the specific embodiment, the present invention is not limited to the embodiment, and various changes can be made without departing from the technical concept of the disclosed invention.

1. A pulse wave detector which is used while being attached to a wrist of a measurement subject, the pulse wave detector comprising:
a body portion which includes a detecting section configured to detect a pulse wave from a radial artery of the measurement subject;
a strip-like band which is configured to secure the body portion to the wrist; and
an attachment auxiliary member which is configured to be attachable to and detachable from the band,
wherein the band is contacted with the wrist through the attachment auxiliary member at an attachment position of the attachment auxiliary member,
wherein the attachment auxiliary member includes, in a contact surface with the wrist, both end portions of the attachment auxiliary member in a short-side direction of the band in a state of being attached to the band, and a concave portion formed between the both end portions to receive a protrusion of the ulna of the wrist,
wherein in a secured state where the body portion is secured to the wrist with the band to which the attachment auxiliary member is attached, the attachment auxiliary member is secured such that the protrusion of the ulna is received in the concave portion and the both end portions are contacted with the wrist.

2. The pulse wave detector according to claim 1,
wherein a surface of the attachment auxiliary member to be contacted with the wrist is curved along a circumferential direction of the wrist.

3. The pulse wave detector according to claim 1,
wherein contact surfaces with the wrist of the both end portions of the attachment auxiliary member are different in position in a direction perpendicular to both the short-side and longitudinal directions of the band.

4. The pulse wave detector according to claim 1,
wherein a surface of the attachment auxiliary member to be contacted with the wrist is covered by an elastic member.

5. A biometric information measurement device comprising:
the pulse wave detector according to claim 1; and
a biometric information calculating section which is configured to calculate biometric information based on the pulse wave detected by the pulse wave detector.

6. An attachment auxiliary member for a pulse wave detector including a body portion which includes a detecting section configured to detect a pulse wave from a radial artery of a wrist of a measurement subject; and a strip-like band which is configured to secure the body portion to the wrist,
wherein the attachment auxiliary member is configured to be attachable to and detachable from the band,
wherein the attachment auxiliary member is contacted with the wrist in place of the band at an attachment position of the attachment auxiliary member,
wherein the attachment auxiliary member includes, in a contact surface with the wrist, both end portions in a short-side direction of the band in a state of being attached to the band, and a concave portion formed between the both end portions to receive a protrusion of the ulna of the wrist,
wherein in a secured state where the body portion is secured to the wrist with the band to which the attachment auxiliary member is attached, the attachment auxiliary member is secured such that the protrusion of the ulna is received in the concave portion and the both end portions are contacted with the wrist.

7. The attachment auxiliary member according to claim 6,
wherein a surface of the attachment auxiliary member to be contacted with the wrist is curved along a circumferential direction of the wrist.

8. The attachment auxiliary member according to claim 6,
wherein contact surfaces with the wrist of the both end portions of the attachment auxiliary member are different in position in a direction perpendicular to both the short-side and longitudinal directions of the band.

9. The attachment auxiliary member according to claim 6,
wherein a surface which is to be contacted with the wrist is covered by an elastic member.
